(19) United States
(12) Patent Application Publication
(10) Pub. No.: US 2017/0240422 A1
(43) Pub. Date: Aug. 24, 2017

(54) TWO-DIMENSIONAL CARBON NITRIDE MATERIAL AND METHOD OF PREPARATION

(71) Applicant: THE UNIVERSITY OF LIVERPOOL, Liverpool (GB)

(72) Inventors: Andrew I. COOPER, Liverpool (GB); Michael J. BOJDYS, Prague (CZ)

(73) Assignee: THE UNIVERSITY OF LIVERPOOL, Liverpool (GB)

(21) Appl. No.: 15/505,510

(22) PCT Filed: Aug. 21, 2014
(86) PCT No.: PCT/GB2014/052568

§ 371 Date: Feb. 21, 2017

(51) Int. Cl. C01B 21/06; H01L 29/24; B01J 6/00

(52) U.S. Cl. CPC C01B 21/0605; B01J 6/008; H01L 29/24

(57) ABSTRACT

Graphitic carbon nitride has been prepared and its structure confirmed by extensive characterization. This material has useful electronic, in particular semiconducting, properties. Crystalline thin films have been prepared. Synthesis may be carried out by condensation of unsaturated carbon- and nitrogen-containing compound(s) in inert solvent such as a salt melt, forming graphitic carbon nitride at a gas-liquid or solid-liquid interface.

The present invention relates to a two-dimensional carbon nitride material, and the synthesis of said material. The material has inherent semiconductor properties and is of particular use in the field of electronics.

Since the advent of single, free-standing 2D sheets of graphite,^{[1]} graphene has been suggested as a promising candidate material for post-silicon electronics.^{[2]} Graphene has the desirable combination of high charge-carrier mobility coupled with high current stability, temperature stability, and thermal conductivity.^{[3]} However, the (semi-)metallic character of graphene and the absence of an electronic bandgap have so far impeded the development of a graphene-based switch.^{[4]} Strategies to open up a graphene bandgap involve single- or multi-step modifications by physical and chemical means.^{[5]} Alternative, simpler routes to silicon-free electronic switches are based on known inherent semiconductors. For example, a field-effect transistor was constructed using single-layer MoS₂ (1.8 eV bandgap) obtained by Scotch tape exfoliation, but this strategy retains the known chemical limitations of MoS₂.^{[6]} It is therefore desirable to complement the electronic properties of the carbon-only graphite/graphene system with a similar material that combines 2D atomic crystallinity and inherent semiconductivity.

The new material discussed here consists exclusively of covalently-linked, sp²-hybridized carbon and nitrogen atoms. It was first postulated by others as “graphitic carbon nitride” (“g-C₃N₄”), by analogy with the structurally related graphite.^{[7]} Over the years, two structural models emerged to account for the geometry and stoichiometry of this as yet hypothetical graphitic carbon nitride. These two models are distinguished by the size of the nitrogen-linked aromatic moieties that make up the individual sheets in the material: one model is based on triazine units (C₃N₃), and the other is based on heptazine units (C₆N₇)^{[8]} Since the 1990s, many attempts at the synthesis of carbon nitride materials have been reported,^{[8]} encompassing chemical vapor deposition (CVD),^{[9]} pyrolysis of nitrogen-rich precursor molecules,^{[10]} shock wave synthesis,^{[11]} and ionothermal condensation.^{[12]} Historically, the existence of a hypothetical, heptazine-based “graphitic carbon nitride (g-C₃N₄)” has been claimed numerous times.^{[10a, 12-13]} Later work revealed these materials to be either polymeric (CNₓH_{y}),^{[14]} or of a poly-(triazine imide)-type,^{[15]} and none of these approaches has yielded a well-defined material of the postulated “g-C₃N₄” structure. The electronic and chemical properties of these materials remain of strong interest: for example, recently a heptazine-based, disordered, more polymeric carbon nitride was shown to facilitate hydrogen evolution from water under visible-light irradiation.^{[16]}

From a first aspect the present invention provides graphitic carbon nitride.

The present inventors are the first to provide an enabling disclosure of this important material. As described in detail below, this material has now been synthesized and fully characterized.

Parts of this specification are taken from the following publication: Algara-Siller, G.; Severin, N.; Chong, S. Y.; Björkman, T.; Palgrave, R. G.; Laybourn, A.; Antenietti, M.; Khimyak, Y. Z.; Krasheninnikov, A. V.; Rabe, J. P.; Kaiser, U.; Cooper, A. I.; Thomas, A.; Bojdys, M. J.: “Triazine-Based Graphitic Carbon Nitride: a Two-Dimensional Semiconductor” Angewandte Chemie International Edition 2014, 53, 7450-7455 (copyright Wiley-VCH Verlag GmbH & Co. KGaA; reproduced with permission), the contents of which (including the Supporting Information) are incorporated herein by reference.

Some previous publications relate to theoretical aspects or predicted properties. Other previous publications disclose materials that are different to true graphitic carbon nitride in that they do not exhibit the same ordered structure and/or they contain elements other than carbon or nitrogen within the repeating units. Yet further previous publications claim to have prepared graphitic carbon nitride, and in many cases use the wording “graphitic carbon nitride” or the label “g-C₃N₄”, whereas subsequent work has shown that the material made was not actually true graphitic carbon nitride. Thus, a large body of prior art exists which incorrectly uses the terms “graphitic carbon nitride” or “g-C₃N₄”. In usage here, the term “carbon nitride” denotes a binary combination of carbon and nitrogen only. The true information content of the prior art does not include true graphitic carbon nitride, in a form that has actually been synthesized, rather than hypothesized or computed, prior to the work of the present inventors. Therefore, any analysis of the prior art needs to go beyond consideration of merely literal statements in paper disclosures where colloquial naming of compounds is commonplace.

Graphitic carbon nitride comprises carbon nitride sheets that exhibit long-range two-dimensional crystalline order.

An alternative definition of the product of the present invention arises from the stoichiometry or empirical formula according to which the material contains sheets of carbon nitride in which there are three carbon atoms for every four nitrogen atoms, and in particular where the repeating units do not contain other elements, at least if one ignores the edges of the carbon nitride layers, and any possible defect structures. This contrasts with certain prior art materials that contain hydrogen in the repeating units, or other materials. Therefore, from a further aspect, the present invention provides graphitic carbon nitride, of the empirical formula C₃N₄, wherein the repeating unit is in the absence of hydrogen. In this context, the skilled person will be aware that any two dimensional material, unless it is an infinite sheet, must of course have edges, and therefore that there may be variation of the material at said edges, for example hydrogen may be present at said edges, albeit in an insignificant amount which does not adversely affect the bulk material properties.

A further definition of the product arises from the nature of the bonding within the two-dimensional carbon nitride structure. The carbon and nitrogen atoms are covalently bonded to each other in a delocalized manner such that the carbon and nitrogen centres are sp²-hybridized. Therefore, from a further aspect, the present invention provides graphitic carbon nitride of the empirical formula C₃N₄ consisting exclusively of covalently-linked, sp²-hybridized, carbon and nitrogen atoms.

The carbon nitride takes the form of crystalline two-dimensional crystals, which exhibit long-range, in-plane order, and the present invention further provides films wherein several two-dimensional crystals may be stacked. For example the films may comprise up to 1000 atomic layers, e.g. up to 500, e.g. up to 100, e.g. up to 50, e.g. up to 20, e.g. up to 10, e.g. up to 5, e.g, 3 atomic layers.

The graphitic carbon nitride may be triazine-based graphitic carbon nitride (TGCN) or heptazine-based graphitic carbon nitride. Both have empirical formula C₃N₄.

These carbon nitride structures are natural semiconductors. Therefore, their inherent properties make them more useful in a greater array of electronic devices than graphene, without needing modification. Nevertheless, the present invention does not exclude the possibility of incorporating doping agents to modify the properties of the material.

The graphitic carbon nitride may be formed on substrates or other materials, For example, graphitic carbon nitride may be formed on insulating materials. Electronic devices in which the graphitic carbon nitride may be used include field-effect transistors and light-emitting diodes, amongst others.

From a further aspect the present invention provides a method of preparing graphitic carbon nitride comprising the condensation of one or more unsaturated, carbon- and nitrogen-containing, compound, in the presence of an inert solvent.

The reaction may be interfacial, such that the graphitic carbon nitride forms at an interface between the solvent (liquid) phase and another phase (solid or gaseous). The reaction may be ionothermal, such that the medium permits reaction at suitable temperature whilst also directing the two dimensional crystal structure of the graphitic carbon nitride. The reaction may be surface-assisted.

The unsaturated carbon- and nitrogen-containing compound may be linear, branched and/or heterocyclic. For example it may comprise one or more of a nitrile, imine, amine, amide, pyrrole, pyridine, isonitrile, cyanuric acid moiety, uric acid moiety or cyamelurine moiety.

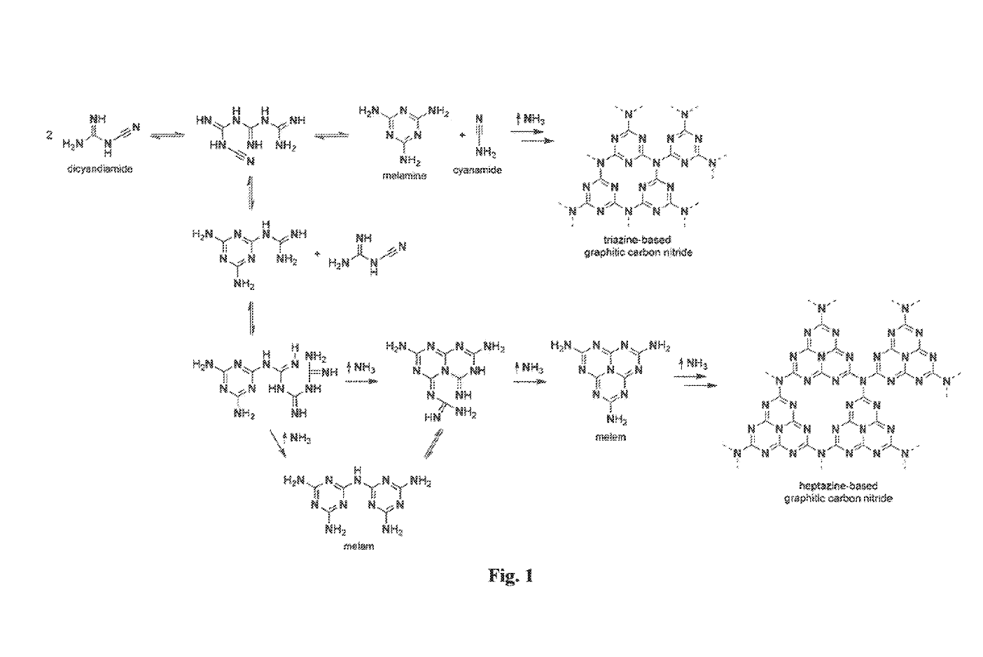

One example of a suitable starting material monomer is dicyandiamine. This is inexpensive and convenient. Other examples of compounds that may be used as suitable reagents include melamine, cyanamide, melam, or melem. Without wishing to be bound by theory, these are believed to be involved in suitable mechanisms leading to the formation of graphitic carbon nitride by condensation and oligomerisation as illustrated in FIG. 1.

The inert solvent may be a molten salt or salt melt, for example those containing one or more metal halides e.g. alkali metal halides, i.e. salts of Li, Na, K, Rb, Cs or Fr with F, Cl, Br or l. Li, Na or K are preferred amongst the alkali metals. Zr or Be halide salts may also be used. Further molten salts may be used, as are known in the art, e.g. in nuclear coolant reactor technology. Mixtures and combinations of salts, e.g. eutectic mixtures, may be used. One, non-limiting, example of a suitable medium is a salt melt of lithium bromide and potassium bromide, in for example a wt % ratio of 30:70 to 70:30, e.g. 40:60 to 60:40, e.g. 45:55 to 55:45, e,g. 50:50 to 54:46, e.g. 51:49 to 53:47, e.g. approximately 52:48. In one non-limiting example the condensation takes place at between 500 and 700° C., e.g. between 550 and 650° C. Other media, mixtures, ratios, and temperatures may be used, so long as they allow reaction to graphitic carbon nitride under inert conditions.

The reaction may take place in a sealed vessel. This can help facilitate the directed synthesis of two-dimensional crystals. The reaction may proceed under autogenous pressure conditions, due to the generation of ammonia or other materials. The reaction may optionally be carried out at a pressure of 5 to 20 bar, e.g, 8 to 18 bar.

The present invention will now be described in further non-limiting detail with reference to the following examples and the figures in which:

FIG. 1 shows a reaction scheme for the formation of graphitic carbon nitride starting from dicyandiamide;

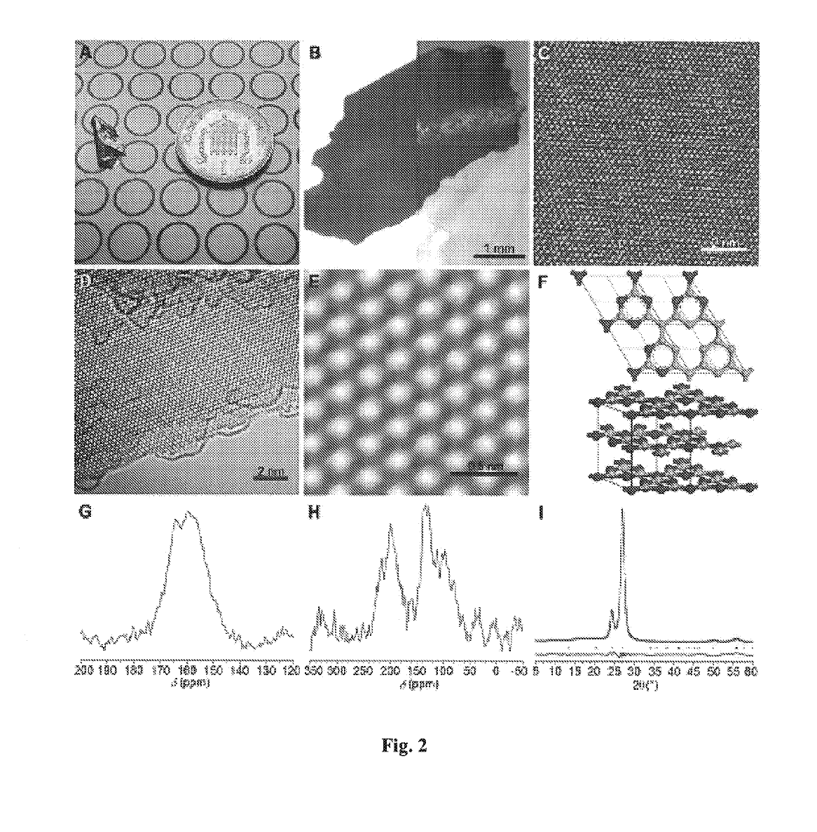

FIG. 2 shows some physical characterization aspects of triazine-based graphitic carbon nitride;

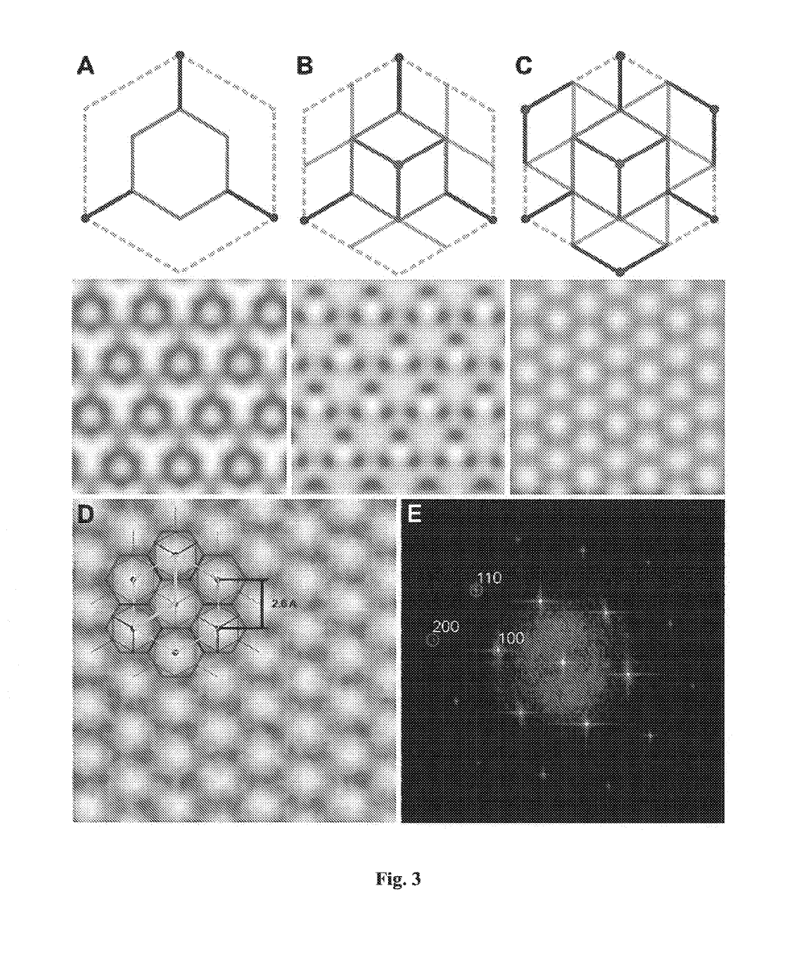

FIG. 3 shows (A, B, C) three possible stacking arrangements of triazine-based graphitic carbon nitride with respective calculated images below, (D) a transmission electron microscopy (TEM) image of TGCN, and (E) a corresponding Fourier transform image; and

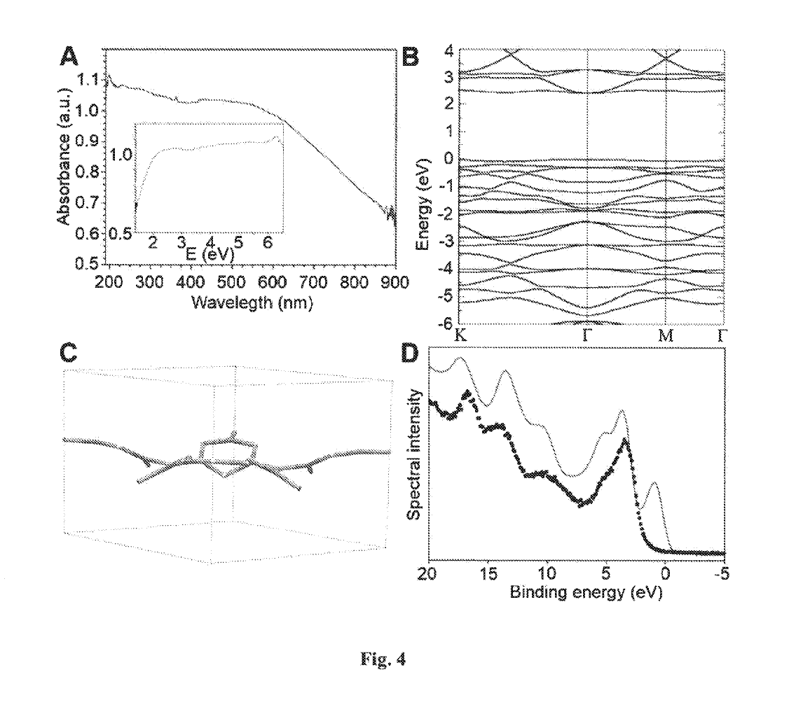

FIG. 4 shows further data in respect of triazine-based graphitic carbon nitride.

### EXAMPLES

Before the present invention, many researchers, over a period of ten years, have tried to synthesize two-dimensional carbon nitride, but have been unsuccessful.

Now, the successful surface-mediated synthesis of 2D crystalline, macroscopic films of graphitic carbon nitride has been achieved.

The material forms interfacially, both at the inherent gas-liquid interface in the reaction and on a quartz glass support.

The principal synthetic procedure is analogous to the previously reported synthesis of poly(triazine imide) with intercalated bromide ions (PTI/130.^{[15a]} In a typical experiment, the monomer dicyandiamide (DCDA) (1 g, 11.90 mmol) is ground with a vacuum-dried, eutectic mixture of LiBr and KBr (15 g; 52:48 wt %, m.p. 348° C.) in a dry environment to prevent adsorption of moisture. The mixture is sealed under vacuum in a quartz glass tube (1=120 mm, outer diameter=30 mm, inner diameter=27 mm) and subjected to the following heating procedure: 1) heating at 40 Kmin⁻¹ to 400° C. (4 h), 2) heating at 40 Kmin⁻¹ to 600° C. (60 h). Safety note: Since ammonia is a by-product of this polycondensation reaction, pressures in the quartz ampoule can reach up to 12 bar, so special care should be taken in handling and opening of the quartz ampoules.

The reaction yields two products: PTI/Br, which is suspended in the liquid eutectic,^{[15a]} and a continuous film of triazine-based, graphitic carbon nitride (TGCN) at the gas-liquid and solid-liquid interface in the reactor. The size of the deposited TGCN flakes scales with the initial concentration of DCDA in the reaction medium, and with the reaction time. Hence, a low initial concentration of the monomeric building blocks (0.5 g DCDA in 15 g LiBr/KBr) yields isolated, transparent flakes of orange-red color (<2 mm), as do shorter reaction times (<24 h). By contrast, a combination of longer reaction times (>48 h) and higher concentrations (1 g DCDA in 15 g LiBr/KBr) of monomer gives macroscopic, shiny flakes that are optically opaque (>10 mm) (FIG. 2A and B).

FIG. 2 shows the physical nature, and characterization, of TGCN made in accordance with the present invention, as follows. A) A single macroscopic flake of TGCN. B) Optical microscopy images of TGCN in transmission (left half) and reflection (right half). C-E) Mechanically cleaved layers of TGCN as imaged by scanning force microscopy (SFM) (C) and by high-resolution TEM (D and E). F) Crystallographic unit cell (a=5.0415(10) Å, c=6.57643(31) Å, space group 187) and AB stacking arrangement of TGCN layers derived from structural refinement. G,H) ¹³C {¹H} magic-angle spinning (MAS) NMR. (MAS rate of 10 kHz) (G) and ¹H-¹⁵N CP/MAS NMR spectra (MAS rate of 5 kHz, reference glycine) (H) of TGCN. I) X-ray analysis of TGCN wherein the observed pattern and the refined profile are substantially overlain as the top line (the bottom line being the difference plot), and Bragg peak positions shown between the two lines.

It is not clear whether the partial pressure of reactive intermediates in the gas phase of the reactor plays a role in the formation of TGCN, because the overall condensation mechanism is accompanied by a release of ammonia (FIG. 1). After cooling, TGCN films can be separated easily from the solidified PTI/Br containing salt block through a simple water washing. The microcrystalline, yellow/brown powder of PTI/Br is suspended in the resulting slurry, while the TGCN flakes float on the surface and can be obtained in pure form by sedimentation and filtration (FIG. 2A and B). TGCN grown at the solid-liquid interface also adheres to the quartz glass support in the reactor and can be peeled, or scratched, away from the surface with relative ease.

We used a combination of transmission electron microscopy (TEM) and scanning force microscopy (SFM) to image the materials and to probe the lateral order of TGCN, and to corroborate historical structural predictions.^{[7b]} Thin sheets of TGCN down to approximately three atomic layers were obtained by mechanical cleavage. TEM images show a hexagonal 2D honeycomb arrangement with a unit-cell of 2.6 Å (FIG. 2E). Under our imaging conditions, the positions of the three coordinated nitrogen atoms of a triazine-based lattice show up as bright areas (FIG. 2D and E). In the stacking model that best reproduces our TEM data (FIG. 2C), the trigonal voids opened up by the three interlinked triazine units are covered by a staggered, graphitic arrangement of subsequent TGCN layers. Unfortunately, no monolayers of TGCN could be obtained by mechanical cleaving. The hexagonal in-plane pattern seen by SFM (a=b=2.78±0.14 Å and a=59.2±2.4°) confirms this repeat of localized electron density (FIG. 2C). We suggest that this lateral repeat corresponds to a hexagonal grid with electronegative nitrogen atoms at its nodes, as seen for the lateral unit cell of TGCN (FIG. 2F). Exhaustive scanning electron microscopy (SEM) imaging and energy-dispersive X-ray (EDX) spectroscopy show a homogeneous, lamellar sample morphology and a composition that comprises carbon and nitrogen in a C₃N₄ ratio. ¹³C and ¹⁵N solid-state NMR spectra (FIG. 2G and H), X-ray photoelectron spectra (XPS) of the C 1 s and N 1 s regions, and electron energy loss spectroscopy (EELS) suggest a material comprised from carbon and nitrogen with the correct hybridization states for an aromatic triazine (C₃N₃)-based structure. The low signal-to-noise ratio in the NMR spectra results from a lack of coupling 1 H environment—as corroborated by elemental analysis, and also from a degree of structural disorder. The quality of the spectra does not allow definitive structural identification, but data suggest one broad ¹³C resonance and two groups of ¹⁵N peaks, both of which are consistent with the structural model of a planar triazine-based material. X-ray diffraction (XRD) analysis confirmed the purity of TGCN, and no diffraction peaks were observed that could be ascribed to the starting material, the salt melt, nor the PTI/Br, which contains heavy halide scatterers (FIG. 21). Following the structural leads from TEM and SFM, we assumed the historical model of “g-C₃N₄”^{[7b]} as an initial guess for structural refinement. This structure is based on a staggered AB arrangement of sheets of nitrogen-bridged triazines (C₃N₃), analogous to graphite (FIG. 2C), and gave reasonable experimental values from Le Bail fitting and constrained structural refinement (a=5.0415(10) Å, c=6.57643(31) Å, space group P6m2, no. 187). Looking at the ab-plane of the refined unit cell, we see a regular grid corresponding to a quarter unit cell giving distances between individual nitrogen atoms of 2.52 Å. The apparent discrepancies in nitrogen-nitrogen distances from TEM (2.60±0.05 Å), SFM (2.78±0.14 Å), XRD (2.52 Å) and DFT (2.39±0.9 Å; min. 2.31 Å, max. 2.66 Å) are intrinsic to these methods, but they give a good overall agreement of 2.57±0.25 Å. The interlayer spacing of 3.28(8) Å (d₀₀₂) is slightly shorter than the gallery height of graphite (3.35 Å) (FIG. 2 I) and in good agreement with other aromatic, discotic systems. The lack of observable peaks for bulk TGCN did not allow a reliable Rietveld refinement of atom positions, or a confident determination of the possible layer stacking arrangements. However, the initial structural model was used to construct three conceivable stacking possibilities: 1) an eclipsed, AA arrangement, in which consecutive sheets are superimposed over each other (FIG. 3A); 2) a staggered, graphite-like AB arrangement with one set of triazine (C₃N₃) units from the first layer always superimposed on top of the voids of the second layer (and their neighbors from the first layer always superimposed on the bridging nitrogens from the second layer) (FIG. 3B); and 3) an ABC stacking, where each triazine (C₃N₃) ring is superimposed on a bridging nitrogen followed by a void (FIG. 3C). Simulated TEM images based on these three models were then compared with the experimental TEM data. An ABC arrangement gave the best fit for the thinnest observed sections of TGCN (FIG. 3). However, stacking disorder in thicker parts of the sample is a possibility, as apparent from the broad (002) peak in the XRD pattern (FIG. 2I). This type of disorder between TGCN layers is known for other discotic systems for which stacking is dominated by non-directional π-π interactions.^{[17]}

Further data is presented in FIG. 4 as follows. A) UV/Vis diffuse-reflectance spectrum with Kubelka-Munk plot (inset) of TGCN. B) DFT calculated band structure for a single sheet of TGCN. C) Corrugated structure of one layer of TGCN found from DFT calculations. D) XPS spectrum of the valence band region of TGCN (dots) and calculated XPS plot for the theoretically determined equilibrium structure (line).

The co-planar arrangement of nitrogen-bridged, aromatic triazine (C₃N₃) units enables extended in-plane delocalization of π-electrons along individual sheets of TGCN, and hence opens up interesting perspectives for electronic applications. The opaque, shiny appearance of bulk TGCN makes optical spectroscopy challenging. However, the onset of an adsorption edge in the red region of the UV/Vis spectrum is discernible (FIG. 4A). Hence, the optical bandgap of TGCN is estimated to be less than 1.6 eV. To corroborate the bandgap properties of TGCN, density functional theory (DFT) calculations were performed using a fully non-local functional that includes van der paals interaction and specifically targets weakly bonded layered systems, (FIG. 4B and C)^{[18]} starting with the original model for “g-C₃N₄”.[7b] The resulting equilibrium structure shows a corrugation of triazine (C₃N₃)-based sheets as observed in previous findings (FIG. 4C)^{[19]} While there is evidence in the literature arid in the present calculations that the actual g-C₃N₄ structure should be non-planar, the actual extent of corrugation/buckling is difficult to access. The lowest energy is found for an AB stacking arrangement, and an interlayer binding energy of 17.6 meV Å⁻² with a minimum interlayer separation of 3.22 Å. The energy differences between AA, AB and ABC stacking configurations are small (max. 14 meV/atom), which indicates that different stacking configurations should be possible, as indicated by XRD and TEM. The band structure for a single layer of the equilibrium structure is shown in FIG. 4C. The single layer bandgap for a free-standing sheet is about 2.4 eV, and it shrinks to 2.0 eV for an AB-stacking arrangement. Since the lowest-energy transition occurs at the G point, TGCN is assumed to be a direct bandgap semiconductor, like polymeric carbon nitride analogues.^{[16]} A comparison of the calculated electronic band structure with the experimental XPS valence band spectrum shows an excellent agreement up to a binding energy of 20 eV, except for the presence of a feature around 1.0 eV in the theoretical spectrum (FIG. 4D). This feature corresponds to 2p orbitals nearly orthogonal to the aromatic plane. Due to very low overlap between the initial pπ state and free photoelectron wavefunctions, such orbitals are known to have anomalously low photoionization cross sections in c-axis-orientated layered materials, such as graphite^{[20]} and h-BN.^{[21]} Thus the absence of this peak in layered TGCN can be rationalized. The calculated valence band spectrum for the unrelaxed, planar structure is significantly different to that observed. Hence, the excellent match between our experimental valence band spectrum and the theoretical spectrum for the relaxed model is more supportive of a corrugated structure. On the whole, combined experimental and computational data, and in particular DFT calculations and XPS measurements, support a corrugated layer structure, although limitations in the various measurement techniques and structural disorder in the TGCN material do not allow us to completely rule out a more planar structure, as found typically in molecular nitrogen-substituted triazines.

From UV/Vis measurements and the correlation of DFT and XPS results, we deduce that TGCN has a bandgap of between 1.6 and 2.0 eV, which places it in the range of small bandgap semiconductors such as Si (1.11 eV), GaAs (1.43 eV), and GO (2.26 eV).^{[22]}

Materials and Methods

Materials. Dicyandiamide (DCDA), lithium bromide and potassium bromide were purchased in their highest-purity form from Sigma-Aldrich and used as received.

Synthesis of TGCN. Dicyandiamide (1 g, 11.90 mmol) was thoroughly ground with 15 g of LiBr/KBr (LiBr/Br dried at 200° C. under vacuum, 52:48 wt %, m.p. 348° C.) in a glove-box (or dry-box) to exclude moisture. The reaction mixture was transferred into a quartz glass ampoule (1=120 mm, o.d.=30 m, i.d.=27 mm) and sealed under vacuum. Subsequently, the reaction mixture was subjected to the following heating procedure: (1) heating at 40 K to 400° C. (4 h), (2) heating at 40 K min⁻¹ to 600° C. (60 h). SAFETY NOTE: Since ammonia is a byproduct of this poly-condensation reaction, pressures in the quartz ampoule can reach at least 12 bar in the configuration described here, so special care should be taken in handling and opening of the quartz ampoules. The actual pressure will of course depend on the relative scale of the ampoule with respect to the reaction contents. After natural cooling, excess salt was removed in boiling distilled water. TGCN was removed via gentle filtration, sieving and by removing flakes of TGCN from the quartz glass. The product was dried thoroughly at 200° C. under vacuum to yield TGCN (92 mg, 0.50 mmol, 12.6% yield) as shiny, dark flakes. Since there is considerable pressure build-up in the quartz glass ampoules during this reaction—leading to loss of ampoules in one out of two cases, an alternative reactor set-up was devised using a stainless steel high-pressure, high-temperature reactor with graphite gaskets and a two-part quartz inlet.

Transmission electron microscopy and image simulation. Electron microscopy was carried out using a Titan 80-300 instrument (FEI) equipped with an imaging-side spherical aberration (CS) corrector operating at an accelerating voltage of 80 kV under Scherzer conditions and with a spherical aberration value of 20 μm. Images were recorded on a CCD (chargecoupled device) with an exposure time of one second per frame and an interval of two seconds between the frames in a particular sequence at a constant electron dose rate of ˜107 electrons nm⁻²s⁻¹.

Scanning force microscopy. SFM was performed under ambient conditions with a Nanoscope 3a (Veeco) instrument equipped with E scanner. Instrument calibration was performed with a standard calibration grid (Veeco) with one micrometer mesh size. Calibration deviations did not exceed 5%, which we also assume to be the calibration error. The imaging was performed in contact mode with silicon nitride cantilevers (Veeco, model: NP-20) with a typical spring constant of 0.12 N/m. To minimize influence of thermal drift, images were acquired with fast scan direction being rotated at different angles. The images were processed with SPIP software (Image Metrology). Averaging of the unit cells gave a=2.77±0.03 Å, b=2.79±0.05 Å and α=59.2±1.7°. Taking into account the instrument calibration error, the unit cell is thus a=b 2.78±0.14 Å and α=59.2±2.4°.

Scanning electron microscopy. SEM imaging of the platelet morphology was achieved using a Hitachi S-4800 cold Field Emission Scanning Electron Microscope (FE-SEM). The dry samples were prepared on 15 mm Hitachi M4 aluminium stubs using either silver dag or an adhesive high purity carbon tab. The FE-SEM measurement scale bar was calibrated using certified SIRA calibration standards. Imaging was conducted at a working distance of 8 mm and a working voltage of 5 kV using a mix of upper and lower secondary electron detectors.

Solid-state NMR. Solid-state NMR spectra were recorded on a Bruker DSX400 spectrometer at room temperature using zirconia MAS rotors. ¹H-¹³C CP/MAS data were recorded using a 4 mm H/X/Y probe head using a MAS rate of 10 kHz. The ¹H π/2 pulse length was 3.1 μs with a recycle delay of 10 s. Two pulse phase modulation (TPPM) heteronuclear dipolar decoupling was used during acquisition.^{[23]} The Hartman-Hahn matching condition was set using hexamethylbenzene (HMB). ¹³C{¹H} MAS were recorded using the same probe head and MAS frequency. A ¹³C π/3 pulse length of 2.6 μs, recycle delay of 20 s and TPPM decoupling were used in acquisition. All ¹³C spectra are referenced to external TMS at 0 ppm. ¹H-¹⁵N CP/MAS spectra were recorded using a 4 mm H/X/Y probe head with a MAS rate of 5 kHz. The ¹H π/2 pulse length was 3.1 μs with a recycle delay of 10 s. Two pulse phase modulation (TPPM) heteronuclear dipolar decoupling was used during acquisition.^{[23]} The Hartman-Hahn matching condition was set using 95% ¹⁵N-Glyciene and contact time of 5 ms was used. All ¹⁵N spectra are referenced to the NH₂ signal of glyciene at 32.5 ppm with respect to NH₃(liq).

Xray photoelectron spectroscopy. XPS measurements were carried out on a Thermo K-alpha spectrometer using monochromated Al Kα radiation with a base pressure of 5×10⁻¹⁰ mbar. Samples were mounted on carbon tape and a focused 400 micron X ray spot was used to ensure signal was only recorded from the sample. An incidence angle of 45° and a take-off angle of 90° were used. A test for beam damage showed no change in any spectra on prolonged exposure to the beam. Charge compensation was carried out using a dual beam electron and Ar+ flood gun. Ion beam etching was carried out in situ using a 1000 eV Ar⁺ beam.

Electron energy loss spectroscopy. Electronic structure measurements were performed using EELS using a GATAN Tridiem image filter on a Philips TEM/STEM CM 200 FEG transmission electron microscope equipped with a field emission gun operating at 200 keV acceleration voltage.

X-ray diffraction. Xray diffraction data was collected in two different set-ups for reproducibility, and diffraction pattern were selected by optimal resolution and signal-to-noise ratio. Laboratory Xray diffraction data were collected in reflection geometry using a PANalytical X'Pert Pro multi-purpose diffractometer (MPD) operating at 40 kV and 40 mA producing Cu Kα radiation and equipped with an open Eulerian cradle. The incident X-ray beam was conditioned with 0.04 rad Soller slits, automatic divergence slit and 5mm mask. The diffracted beam passed through 0.04 rad Soller slits and a parallel plate collimator. Data were collected over the range 4≦2θ≦90° with a step size of 0.02° over 19 h. Structural refinement and Le Bail fitting was carried out using the TOPAS-Academic software.^{[24]} For the structural refinement of the P-6m2 Teter model against the experimental diffraction data, geometric restraints were applied to all bond distances and angles. The asymmetric unit consisted of two carbon atoms and four independent nitrogen atoms. One half of the asymmetric unit, i.e. CN₂ was constrained to lie on the mirror plane at x,y,0, while the z-coordinates of the other half were fixed to position it on the (x,y,½) plane. One nitrogen on each mirror plane was fixed on a high symmetry -6m2 special position. The refinement of x and y coordinates of all other atoms were constrained to mm2 positions,

Infrared spectroscopy. Fourier transform infrared (FT-IR) measurements were carried out on a Bio-Rad FTS-6000 system in attenuated total reflection (ATR) setup. FTIR spectra of bulk samples were recorded at ambient temperature.

Raman spectroscopy. Raman spectra were recorded on a Renishaw spectrometer and excitation wavelength of 488 nm using freshly cleaved TGCN and single-layer graphene (SLG) for comparison. SLG was deposited on mica substrate (Ratan mica exports, V1 quality), and TGCN was measured on adhesive tape.

Density functional theory methods. DFT calculations were performed with the projector augmented wave method^{[25,26]} as implemented in the VASP package.^{[27,28]} Relaxations were done with a gamma-centred k-point mesh giving a k-point density of 0.2 Å⁻¹ and with an energy cut-off for the plane wave basis of 600 eV. Initially, relaxations were performed using the PBE functional^{[28]} for a single layer for all surface supercells up to a 3×3 supercells of the “g-C₃N₄” cell. The lowest energy was obtained for the (√3×√3)R30° supercell (degenerate with the 3×3 supercell, which contains three such structures), which was then used as basis for relaxation of the 3D structure using the AM05-VV10sol)functional.^{[30]} Since the implementation of the non-local van der Waals density functional²⁵ does not support calculation of the stress tensor, relaxations of the bulk 3D structure were done by direct minimization of the total energy with respect to variations of the lattice vectors using the Nelder-Mead downhill simplex algorithm, while allowing for full relaxation of internal forces in each step. Different stacking of the flat starting-structure with small random distortions of the atomic positions were allowed to relax to the lowest energy configuration and in all cases the same inplane structure was found as in the PBE relaxation of a single layer, thus rang out the possibility that the equilibrium geometry is strongly dependent on the choice of functional in this case. The lowest-energy configuration found was an AB stacking of corrugated planes (FIG. 2, C). This configuration is lower in energy by 4.5 meV/atom compared to the ABC stacking (FIG. 3, C) and lower by 9.7 meV/atom compared to AA stacking (FIG. 3, A). The least energetically favourable stacking arrangement examined was elevated by 14 meV/atom compared to the AB stacking.

In summary, a triazine-based, graphitic carbon nitride that was predicted in 1996 has now been successfully synthesized. Because of its direct, narrow bandgap, TGCN provides new possibilities for post-silicon electronic devices. In particular, the crystallization of semiconducting TGCN at the solid-liquid interface on insulating quartz offers potential for a practically relevant device-like adaptation.

[1] K. S. Novoselov, D. Jiang, F. Schedin, T. J. Booth, V. V. Khotkevich, S. V. Morozov, A. K. Geim, Proc. Natl. Acad. Sci. USA 2005, 102, 10451-10453.

[2] a) S. Hertel, D. Waldmann, J. Jobst, A. Albert, M. Albrecht, S. Reshanov, A. Schçner, M. Krieger, H. B. Weber, Nat. Common. 2012, 3, 957; b) A. K. Geim, K. S. Novoselov, Nat. Mater. 2007, 6, 183-191; c) B. Standley, W. Bao, H. Zhang, J. Bruck, C. N. Lau, M. Bockrath, Nano Lett. 2008, 8, 3345-3349.

[3] a) A. S. Mayorov, R. V. Gorbachev, S. V. Morozov, L. Britnell, R. Jalil, L. A. Ponomarenko, P. Blake, K. S. Novoselov, K. Watanabe, T. Taniguchi, A. K. Geim, Nano Lett. 2011, 11, 2396-2399; b) S. Hertel, F. Kisslinger, J. Jobst, D. Waldmann, M. Krieger, H. B. Weber, Appl. Phys. Lett. 2011, 98, 212109; c) Y. Zhang, Y.-W. Tan, H. L. Stormer, P. Kim, Nature 2005, 438, 201-204.

[4] F. Schwierz, Nat. Nanotechnol. 2010, 5, 487-496.

[5] a) T. Ohta., A. Bostwick, T. Seyller, K. Horn, E. Rotenberg, Science 2006, 313, 951-954; b) J. B. Oostinga, H. B. Heersche, X. L. Liu, A. F. Morpurgo, L. M. K. Vandersypen, Nat. Mater. 2008, 7, 151-157; c) C. Berger, Z. Song, X. Li, X. Wu, N. Brown, C. Naud, D. Mayou, T. Li, J. Hass, A. N. Marchenkov, E. H. Conrad, P. N. First, W. A. de Heer, Science 2006, 312, 1191-1196; d) F. Withers, M. Dubois, A. K. Savchenko, Phys. Rev. B 2010, 82, 073403.

[6] a) K. F. Mak, C. Lee, J. Hone, J. Shan, T. F. Heinz, Phys. Rev. Lett. 2010, 105, 136805; b) B. Radisavljevic, A. Radenovic, J. Brivio, V. Giacometti, A. Kis, Nat. Nanotechnol. 2011, 6, 147-150.

[7] a) A. Y. Liu, R. M. Wentzcovitch, Phys. Rev. B 1994, 50, 10362-10365; b) D. M. Teter, R. J. Hetriley, Science 1996, 271, 53-55.

[8] E. Kroke, M. Schwarz, Coord. Chem. Rev. 2004, 248, 493-532.

[9] J. Kouvetakis, A. Bandari, M. Todd, B. Wilkens, N. Cave, Chem. Mater. 1994, 6, 811-814.

[10] a) T. Sekine, H. Kanda, Y. Bando, M. Yokoyama, K. Hojou, J. Mater. Sci. Lett. 1990, 9, 1376-1378; h) A. Thomas, A. Fischer, F. Goettmann, M. Antonietti, J. O. Muller, R. Schlogl, J. M. Carlsson, J. Mater. Chem. 2008, 18, 4893-4908.

[11] M. R. Wixom, J. Am. Ceram. Soc. 1990, 73, 1973-1978.

[12] M. J. Bojdys, J. O. Mueller, M. Antonietti, A. Thomas. Chem. Eur. J. 2008, 14, 8177-8182.

[13] a) B. V. Lotsch, W. Schnick, Chem. Mater. 2005, 17, 3976-3982; b) X. Li, J. Zhang, L. Shen, Y. Ma, W. Lei, Q. Cui, G. Zou, Appl. Phys. A 2009, 94, 387-392.

[14] B. V. Lotsch, W. Schnick, Chem. Eur. J. 2007, 13, 4956-4968.

[15] a) S. Y. Chong, J. T. A. Jones, Y. Z. Khimyak, A. I. Cooper, A. Thomas, M. Antonietti, M. J. Bojdys, J. Mater. Chem. A 2013, 1, 1102-1107; b) E. Wimhier, M. Dçblinger, D. Gunzelmann, J. Senker, B. V. Lotsch, W. Schnick, Chem. Eur. J. 2011, 17, 3213-3221.

[16] X. C. Wang, K. Maeda, A. Thomas, K. Takanabe, G. Xin, J. M. Carlsson, K. Domen, M. Antonietti, Nat. Mater. 2009, 8, 76-80.

[17] A. P. Cote, A. I. Benin, N.W. Ockwig, M. O_Keeffe, A. J. Matzger, O. M. Yaghi, Science 2005, 310, 1166-1170.

[18] T. Bjorkman, Phys. Rev. B 2012, 86, 165109.

[19] a) Y. Miyamoto, M. L. Cohen, S. G. Louie, Solid State Commun. 1997, 102, 605-608; b) D. T. Vodak, K. Kim, L. Iordanidis, P. G. Rasmussen, A. J. Matzger, O. M. Yaghi, Chem. Eur. J. 2003, 9, 4197-4201.

[20] U. Berg, G. Drager, O. Brummer, Phys. Status Solidi B 1976, 74, 341-348.

[21] E. Tegeler, N. Kosuch, G. Wiech, A. Faessler, Phys. Status Solidi B 1979, 91, 223-231.

[22] B. G. Streetman, B.S., Solid State Electronic Devices, 6 ed., Pearson, 1999,

[23] Bennett, A. E., Rienstra, C. M., Auger, M., Lakshmi, K. V. & Griffin, R. G. HETERONUCLEAR DECOUPLING IN ROTATING SOLIDS. Journal of Chemical Physics 103, 6951-6958, doi: 10.1063/1,470372 (1995).

[24] A. A. Coelho, TOPAS Academic, version 4.1, 2007; http://www.topas-academic.net.

[25] Blochl, P. E. PROJECTOR AUGMENTED-WAVE METHOD. Phys. Rev. B 50, 17953-17979, doi:10.1103/PhysRevB.50.17953 (1994).

[26] Kresse, G. & Joubert, D. From ultrasoft pseudopotentials to the projector augmented-wave method. Phys, Rev. B 59, 1758-1775, doi:10.1103/PhysRevB.59.1758 (1999).

[27] Kresse, G. & Hafner, J. AB-INITIO MOLECULAR-DYNAMICS SIMULATION OF THE LIQUID-METAL AMORPHOUS-SEMICONDUCTOR TRANSITION IN GERMANIUM. Phys. Rev. B 49, 14251-14269, doi:10.1103/PhysRevB.49.14251 (1994).

[28] Kresse, G. & Furthmuller, J. Efficiency of ab-initio total energy calculations for metals and semiconductors using a plane-wave basis set. Computational Materials Science 6, 15-50, doi:10.1016/0927-0256(96)00008-0 (1996).

[29] Perdew, J. P., Burke, K. & Ernzerhof, M. Generalized gradient approximation made simple. Phys. Rev. Lett 77, 3865-3868. doi:10.1103/PhysRevLett.77.3865 (1996).

[30] Gulans, A., Puska, M. J. & Nieminen, R. M. Linear-sealing self-consistent implementation of the van der Waals density functional. Phys. Rev. B 79, doi:10.1103/PhysRevB.79.201105 (2009).

1. Graphitic carbon nitride.

2. Graphitic carbon nitride as claimed in claim 1, of the empirical formula C₃N₄, wherein the repeating unit is in the absence of hydrogen.

3. Graphitic carbon nitride as claimed in claim 1, of the empirical formula C₃N₄consisting exclusively of covalently-linked, sp²-hybridized carbon and nitrogen atoms.

4. Graphitic carbon nitride as claimed in any preceding claim in the form of a film comprising stacked two-dimensional crystals of C₃N₄.

5. Graphitic carbon nitride as claimed in claim 4 wherein the film comprises up to 1000 atomic layers.

6. Graphitic carbon nitride as claimed in claim 4 wherein the film comprises up to 100 atomic layers.

7. Graphitic carbon nitride as claimed in claim 4 wherein the film comprises 3 atomic layers.

8. Graphitic carbon nitride as claimed in any preceding claim wherein the graphitic carbon nitride is triazine-based graphitic carbon nitride.

9. Graphitic carbon nitride as claimed in any preceding claim, further comprising a doping agent.

10. Product or device comprising graphitic carbon nitride as claimed in any preceding claim, on a substrate, and/or in combination with one or more layer of other material.

11. Use of graphitic carbon nitride, as claimed in any of claims 1 to 9, in electronics.

12. Use of graphitic carbon nitride, as claimed in any of claims 1 to 9, as a semiconductor.

13. Method of preparing graphitic carbon nitride as claimed in any of claims 1 to 9 comprising the condensation of one or more unsaturated, carbon- and nitrogen-containing, compound, in the presence of an inert solvent.

14. Method as claimed in claim 13 comprising surface-assisted synthesis such that the graphitic carbon nitride forms at a solid-liquid interface of or within a reactor, or at a gas-liquid interface.

15. Method as claimed in claim 13 or claim 14 wherein said unsaturated, carbon- and nitrogen-containing, compound comprises one or more of a nitrile, imine, amine, amide, pyrrole, pyridine, isonitrile, cyanuric acid moiety, uric acid moiety or cyamelurine moiety.

16. Method as claimed in claim 13 or claim 14 wherein said unsaturated, carbon- and nitrogen-containing, compound is dicyandiamide.

17. Method as claimed in claim 13 or claim 14 wherein said unsaturated, carbon- and nitrogen-containing, compound is one of more of melamine, cyanamide, melam, or melem.

18. Method as claimed in any of claims 13 to 17 wherein said inert solvent is a molten salt.

19. Method as claimed in any of claims 13 to 17 wherein said inert solvent is a salt melt comprising one or more alkali halide.

20. Method as claimed in any of claims 13 to 17 wherein said inert solvent is a salt melt comprising a eutectic mixture of lithium bromide and potassium bromide.

21. Method as claimed in any of claims 13 to 20 wherein condensation is carried out a temperature of between 500 and 700° C.

22. Method as claimed in any of claims 13 to 21 wherein the reaction is carried out in a sealed vessel.

23. Graphitic carbon nitride obtained by a method as claimed in any of claims 13 to 22.

24. Apparatus for preparing graphitic carbon nitride in accordance with any preceding claim.
